(19) United States
(12) Patent Application Publication
(10) Pub. No.: US 2007/0287084 A1
(43) Pub. Date: Dec. 13, 2007

(54) Light Sensitive Organic Photoconductor

(76) Inventors: Mark Thomas Bellino, Loveland, CO (US); David Glenn Black, Longmont, CO (US); Weimei Luo, Louisville, CO (US); Dai Q. Nguyen, Platteville, CO (US); Scott Daniel Reeves, Louisville, CO (US)

Correspondence Address: LEXMARK INTERNATIONAL, INC.;INTELLECTUAL PROPERTY LAW DEPARTMENT, 740 WEST NEW CIRCLE ROAD, BLDG. 082-1, LEXINGTON, KY, 40550-0999, US

(21) Appl. No.: 11/422,781

(22) Filed: Jun. 7, 2006

(51) Int. Cl. G03G 15/02; C07D 487/22

(52) U.S. Cl. 430/59.5; 540/140

(57) ABSTRACT

An electrophotoconductive element which may be irradiated with laser light having a wavelength (λ) of about 350-500 nm which contains titanylphthalocyanine (TiOPC), which may be polymorphic. The electrophotoconductive element may therefore include an electroconductive support, a charge generation layer containing TiOPC, a charge transport layer and a source of laser light having a wavelength of about 350-500 nm. The charge transport layer may also exhibit light transmitting properties over the indicated laser wavelengths.

### FIELD OF THE INVENTION

The present invention relates to photoconductors with sensitive to certain wavelengths of light, which may be employed in laminate type organic photoconductors. The photoconductors may be selectively irradiated with light at selected wavelengths to provide latent image formation that may then be utilized in an electrophotographic imaging systems.

### BACKGROUND OF THE INVENTION

A photoconductive device for an electrophotographic imaging system may include a conductive substrate coated with a charge generation layer (CGL) which in turn may be coated with a charge transport layer (CTL). Typically, such devices may be configured to have relatively useful levels of sensitivity to a relatively long wavelength region of approximately 700-800 nm. Accordingly, such devices may rely upon charge generation materials which, while sensitive to wavelengths of 700-800 nm, do not generally have absorption bands at about 400-500 nm. However, photogeneration using a lower wavelength may be desirable as shorter wavelength irradiation may provide relatively higher print resolution.

### SUMMARY OF THE INVENTION

In a first exemplary embodiment, the present invention relates to an electrophotoconductive element which may be irradiated with laser light having a wavelength (λ) of about 350-500 nm which contains titanylphthalocyanine (TiOPC). In another exemplary embodiment, the present invention relates to an electrophotographic photoconductor system comprising an electroconductive support, a charge generation layer (CGL), a charge transport layer (CTL) and a source of light having a wavelength of about 350-500 nm. The charge transport layer may exhibit light transmitting properties with respect to such laser light wherein the charge generation layer may contain a titanylphthalocyanine (TiOPC) photoconductor capable of absorbing the laser light at the indicated wavelengths. In yet another exemplary embodiment the present invention relates to a method of forming an image in an electrophotographic device. The method may include the steps of providing an electrophotoconductive element comprising an electroconductive support, a charge generation layer and a charge transport layer. The charge generation layer may include a titanylphthalocyanine (TiOPC) photoconductor that is capable of absorbing laser light having a wavelength of about 350-500 nm. The method may then include the step of charging the element and exposing to laser light restricted to a wavelength of about 350-500 nm with the formation of an electrostatic latent image.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 illustrates a cross section of an exemplary layered photoconductive imaging device;

FIG. 2 illustrates a chemical structure for titanylphthalocyanine;

FIG. 3 illustrates an X-ray diffraction pattern for Type I TiOPC;

FIG. 4 illustrates an X-ray diffraction pattern for Type IV TiOPC;

FIG. 5 illustrates the solution and solid state optical absorption of TiOPC;

FIG. 6 illustrates percent transmittance spectra for bisphenol-Z-polycarbonate (PCZ); N,N′-diphenyl-N,N′-di(m-tolyl)-p-benzidene (TPD); tritolylamine (TTA); and 1,1-bis(di-4-tolylaminophenyl)cyclohexane (TAPC);

FIG. 7 illustrates a schematic of the applied potential on a sample photoconductor surface vs. time; and

FIG. 8 illustrates the discharge of potential vs. time for Type IV TiOPC and a Type I TiOPC/Type IV TiOPC as measured on a Rotating Disk Electrometer.

### DETAILED DESCRIPTION

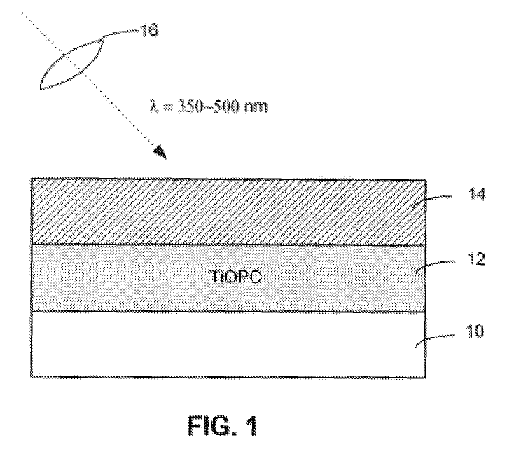

FIG. 1 provides a cross-sectional view of an exemplary layer configuration in a electrophotographic photosensitive device. As illustrated, the device may have a conductive substrate 10, a charge generation layer (CGL) 12 and a charge transport layer (CTL) 14 formed thereon. It can be appreciated that one may also include a protective layer on the CTL. The conductive substrate 10 may be formed from a metal or a metallic alloy, e.g. aluminum, an aluminum alloy, stainless steel, copper, etc. A laser light source 16 may also be provided.

The charge generating layer may contain a charge generating material and may be formed by dispersing the charge generating material into a suitable binder and coating the dispersion on a conductive substrate. One suitable procedure for forming a charge generation layer may be found in U.S. Pat. No. 6,787,276, whose teachings are incorporated by reference. The CGL may also be formed by a relatively dry process such as deposition, sputtering or a CVD process. The binder may be selected from a variety of binder resins, including polymeric based materials. For example, it may include polycarbonate resins, polyester resins, polyarylate resins, butyral resins, polystyrene resins, poly(vinyl acetal) resins, diallyl phthalate resins, acrylic resins, methacrylic resins, vinyl acetate resins, phenol resins, silicone resins, polysulfone resins, styrene-butadiene resins, alkyd resins, epoxy resins, urea resins, vinyl chloride-vinyl acetate resins, either alone or in combination.

The charge generation layer herein may contain the binding resin and charge generating compound in suitable proportion to provide a charge generating effect. For example, the CGL may contain charge generation compound in an amount from about 10% to about 90% by weight (wt.), including all values and increments therein. In addition, the thickness of the CGL may fall within the range of about 0.05 to about 5.0 microns, including all values and ranges therein. It should be noted that the thickness of the CGL may be conveniently monitored by tracking the optical density using a Macbeth TR524 densitometer.

The charge transport layer may contain charge transport compound and similarly contain binders of the type noted above for the charge generation layer. Charge transport compounds suitable for use in the CTL of the present invention may include those compound that are capable of supporting the injection of photogenerated holes and electrons from the CGL, which may then allow for the transport of these holes or electrons through the CTL to selective discharge a surface charge. Suitable charge transport compounds may again be found in U.S. Pat. No. 6,787,276. In addition, the CTL may include charge transport compound(s) in an amount from about 5-60 weight percent, based upon the weight of the charge transport layer, including all values and increments therein.

The laser light source of the present invention may include a laser capable of supplying light having an oscillation wavelength of between 350-500 nm, including all values and increments therein. Accordingly, the laser light source may also include a laser that has a restricted output to such oscillation wavelengths. It may also be appreciated that photogeneration utilizing such relatively lower wavelength source may now provide an advantage over relatively longer wavelength generation. This may be appreciated by a consideration of spot diameter, which diameter may recognized to scale linearly with wavelength according to the following formula:

d_{spot diameter}=(π/4)(λf/D)

wherein λ corresponds to laser beam wavelength, f corresponds to the focal length and D corresponds to the diameter of the lens. Accordingly, by way of the present invention, the use of a laser light source having oscillation wavelengths between about 350-500 nm, in combination with a titanylphthalocyanine (TiOPC) photoconductor capable of absorbing such laser light, may yield higher print resolution. In addition, other potential advantages may include reduced energy requirements and lower relative cost of operation. Therefore, suitable lasers that may be contemplated herein may include GaN and AlGaInN lasers, which may provide emissions centered around 400 nm.

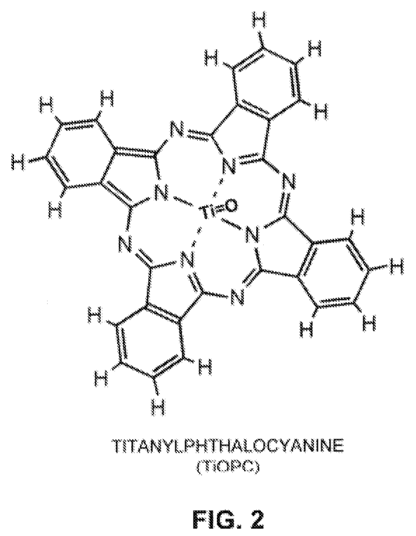

The structure of titanylphthalocyanine (TiOPC) may be illustrated as shown in FIG. 2, which has a molecular formula C₃₂H₁₆N₈OTi and a molecular weight (MW) of 576.39. As alluded to above, TiOPC is capable of absorbing laser light between about 350-500 nm, including all values and increments therein. Accordingly, the TiOPC may absorb between 360-480 nm, or between 370-470 nm, etc. In addition, the TiOPC that may be employed herein may be polymorphic and thereby capable of forming different crystalline forms, which may be identified by X-ray diffraction patterns. Such different crystalline forms may include what may be identified as Type I TiOPC and/or Type IV TiOPC, and either one or a mixture of such crystalline forms may be employed as a charge generation compound within the charge generation layer described above. For example, one crystalline form of TiOPC (e.g. Type I TiOPC) may be present at levels between 1-99% (wt.) and a second crystalline form (e.g. Type IV TiOPC) may be present at level between 99-1% (wt.), including all values and increments therein.

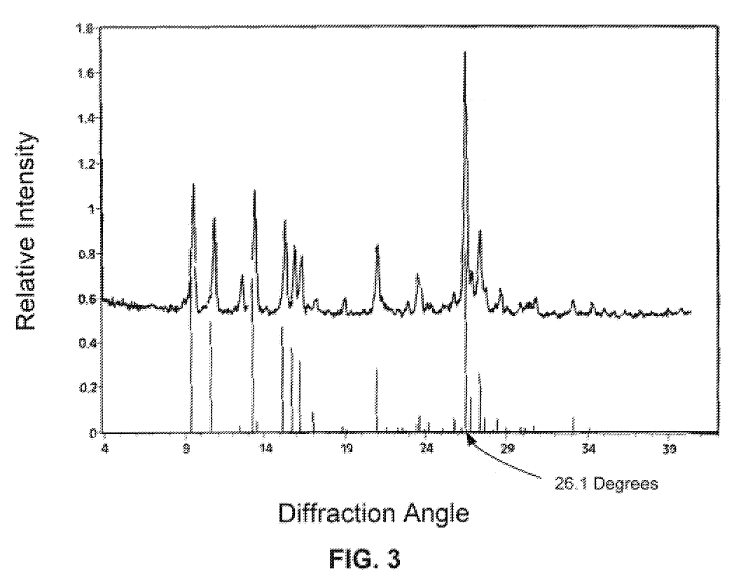

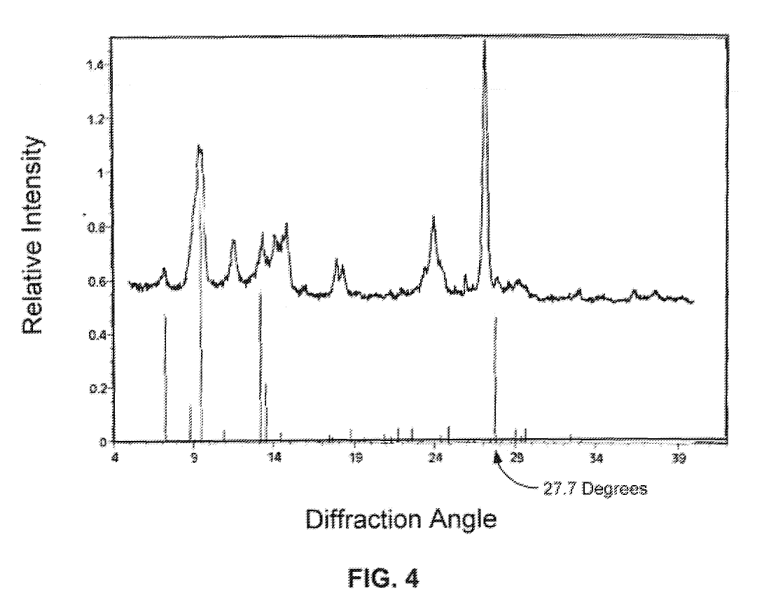

Turning then to FIG. 3, Type I TiOPC may be identified as having a plurality of diffraction peaks when measured by a Phillips Powder Diffractometer with scanning from 5-45 degrees two theta (2θ) at 2 degrees/minute utilizing Cu K-alpha radiation. The strongest intensity of the diffracted X-rays may be seen to occur between 26.0-27.0 degrees, and more specifically at 26.5 degrees, +/−0.4 degrees. Accordingly, the strongest intensity for Type I TiOPC may be more specifically observed between 26.1-26.9 degrees. As next shown in FIG. 4, Type IV TiOPC may indicate an X-ray diffraction pattern, again with a plurality of diffraction peaks, with the strongest intensity of diffracted X-rays observed between 27.0-28.5 degrees. More specifically, Type IV TiOPC may indicate the strongest intensity of diffracted X-rays at 27.7 degrees, +/−0.4 degrees. Accordingly, the strongest intensity for Type IV TiOPC may be more specifically observed between 27.3-28.1 degrees.

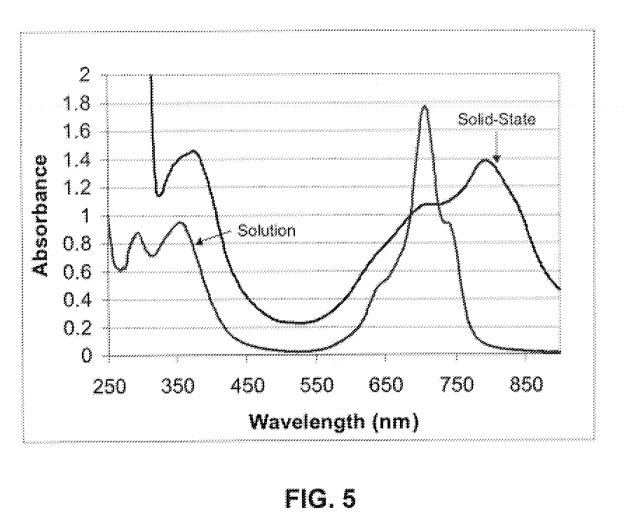

TiOPC may also be characterized by a solution UV spectrum by dissolving, e.g., Type IV titanylphthalocyanine in a mixture of trifluoroacetic acid/dichloromethane (10/90 v/v). The solid state UV visible absorbance may then be recorded by coating the Type IV titanylphthalocyanine dispersion onto a transparent MYLAR® sleeve. The optical absorption spectra may then be recorded utilizing a Genesys 2 Spectrophotometer, available from Thermospectronics, Inc. As shown in FIG. 5, the solution and solid state optical absorption properties of titanylphthalocyanine are different. A relatively sharp peak at about 670 nm (Q band) and a broader peak below 400 nm (Secret band) dominate the solution absorbance. The solid state absorbance spectrum demonstrates a broader Q band and a maximum absorbance that has shifted to 780 nm. However, the shape of the Soret band appears relatively similar to that of the solution spectrum at around 400 nm, and it may be appreciated that the intensity of the absorbance may be dependent upon the concentration of the TiOPC. In any event, and among other things, FIG. 5 identifies the ability herein of titanylphthalocyanine to serve as a charge generation compound suitable to respond to laser light having a wavelengths of about 350-500 nm.

Expanding next on the above referenced charge transport molecules, such molecules may include those which transmit 25% or more of the laser light having wavelengths of about 350-500 nm, including all values and increments between 25-100%. In this fashion, more efficient delivery of such laser light may reach the charge generation layer. In addition, such transmission need not apply to the entire wavelength range of 359-500 nm and such percent transmission may be isolated to any given wavelength value or range of wavelengths between 350-500 nm.

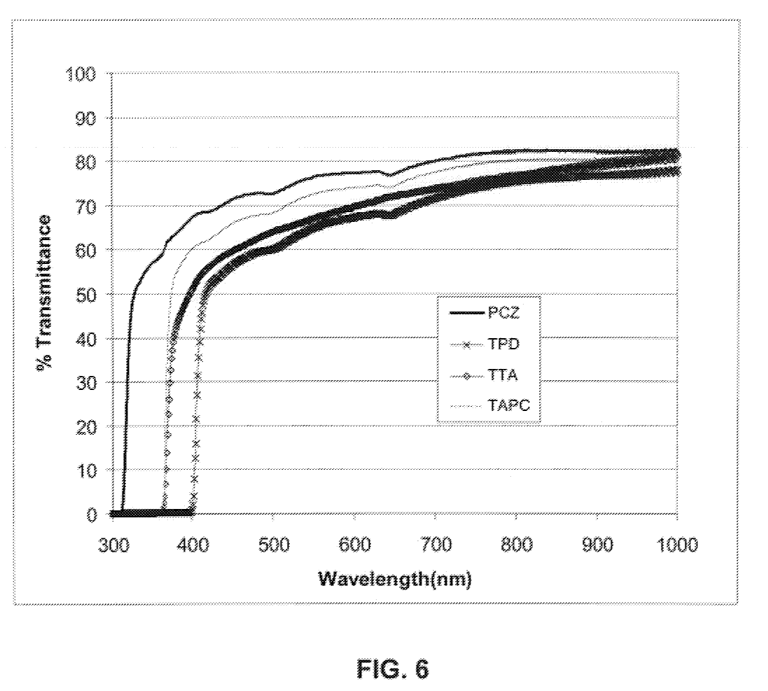

By way of example, the percent transmittance of charge transport binder and three (3) suitable charge transport molecules was examined and evaluated by first applying 5′×10′ sheets of MYLAR® onto a cylindrical aluminum substrate. Charge transport solutions were then prepared (at about 20% solids) by dissolving 25 parts of charge transport molecule and 75 parts polycarbonate Z in a solvent blend of THF/1,4-dioxane (75/25 w/w). The resulting solutions were then coated over the MYLAR® sleeve via dip-coated and dried at about 100° C. for about one hour. The coating thickness was adjusted to about 25 microns by altering the coating speed. The percent transmittance spectra may then be recorded utilizing a Genesys 2 Spectrophotometer, available from Thermospectronics, Inc. Exemplary charge transport molecules include N,N′-diphenyl-N,N′-di(m-talyl)-p-benzidene (TPD); 1,1-bis(di-4-tolylaminophenyl)cyclohexane (TAPC) and tritolyamine (TTA). Bisphenol-Z-polycarbonate (PCZ) was coated pure and represents one exemplary binder. The percent transmittance spectra is illustrated in FIG. 6. As can be seen, the percent transmittance spectra confirm that PCZ, TTA and TAPC begin to transmit at wavelengths at or greater than about 300 nm, and at about 350 nm-375 nm, the transmittance of TAPC and TTA is at least about 25%. The transmittance of TPD begins at about 400 nm.

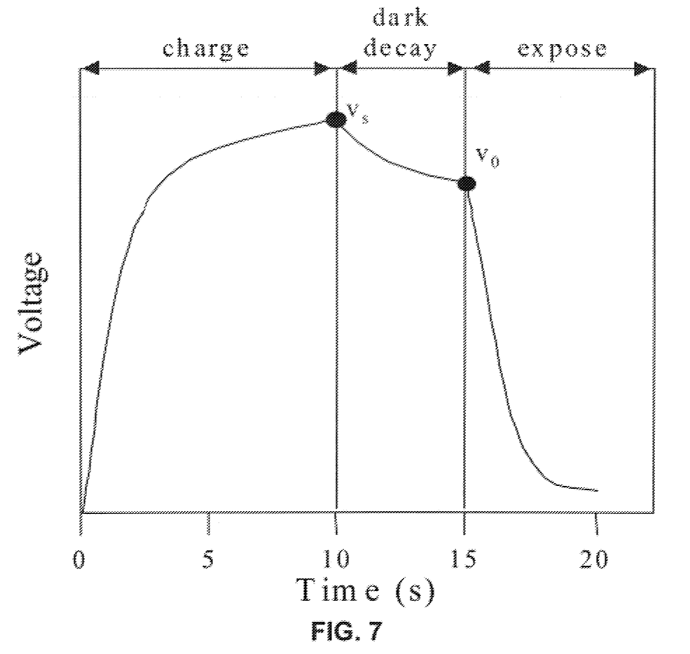

Type IV TiOPC and 85/15 mixture of Type IV TiOPC as a charge generation layer in a suitable binder were next evaluated on a Monroe Static Charge Analyzer Model 270A, also known as a rotating disk electrometer (RDE) utilizing a charge current of 100 μA and a 405 nm broadband filter (68 nm bandwidth at half maximum). The samples, which consist of dual layer photoconductor coated over 5′×10′ aluminized MYLAR ® sheets (as noted more fully below) may be cut into round disks. The photoconductor may then be negatively charged by the charge corona. The potential on the photoconductor surface may then be recorded and plotted. The potential at the end of the charging period is identified as Vs. The photoconductor is then allowed to dark decay for a predetermined time (5 sec). The potential at the end of the dark decay is identified as Vo. The photoconductor may then be exposed to light and discharged. A schematic of this process is illustrated in FIG. 7.

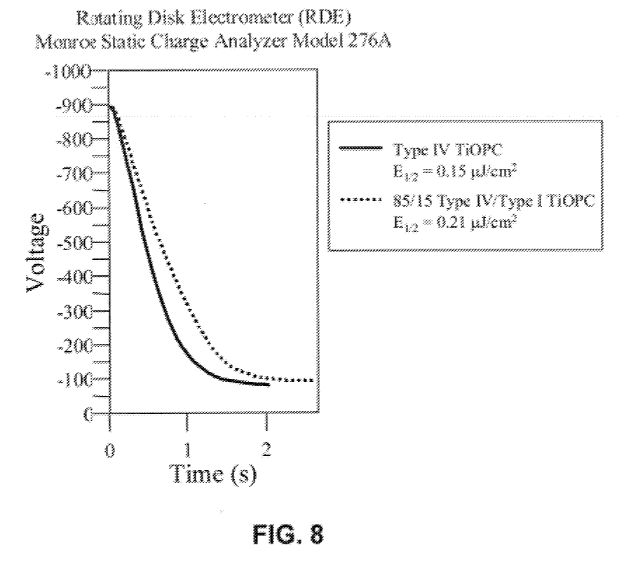

The dual-layer samples for testing were again prepared by first forming a charge generation layer on the 5″×10″ MYLAR®, and as alluded above, the samples included one containing Type IV TiOPC and one containing an 85/15 Type IV/Type I TiOPC. A charge transport layer was then formed thereon, by again preparing a charge transport solution (20 percent solids) by dissolving 35 parts TTA, 5 parts TAPC and 60 parts polycarbonate Z in a solvent blend of THF/1,4-dioxane (75/25 W/W). The solutions were then coated on the charge generation layer via dip coating and dried at 100° C. for one hour. Coating thickness of the CTL was about 25 microns. The samples for use in the rotating disk electrometer were then cut into circles of about 1″ diameter. Conductive metallic (e.g. silver) paint may then be placed on an edge of the circle for electrical testing. The voltage versus exposure time curves for dispersions containing Type IV TiOPC and the 85/15 mixture of Type IV TiOPC/Type I TiOPC are shown in FIG. 8, which more specifically shows discharge curves for TiOPC samples with a charge transport layer containing 35/5 TTA/TAPC in PCZ300 at 405 nm. As can be seen, the Type IV TiOPC and the 85/15 mixture of Type IV TiOPC/Type I TiOPC both decay to approximately the same final voltage, but the Type IV TiOPC does so faster, as indicated by the lower value for E_{1/2}. It Is clear, however, that the discharge may be controlled by the feature of TiOPC polymorphism which thereby may provide flexibility in control of image development when employed as a photoconductive element in an electrophotographic printer. Or, stated another way, by regulating the presence and concentration of different crystalline forms of TiOPC within a photoconductive element, photoconductive discharge and image development may now be optimized for a given photoconductive/laser combination.

A number of implementations have been described. Nevertheless, it will be understood that various modifications may be made. Accordingly, other implementations are within the scope of the following claims.

What is claimed is:

1. An electrophotoconductive element irradiated with laser light having a wavelength of about 350-500 nm, comprising a titanylphthalocyanine (TiOPC) photoconductor capable of absorbing said laser light.

2. The electrophotoconductive element of claim 1, wherein said titanylphthalocyanine absorbs within the wavelengths of about 360 nm-480 nm.

3. The electrophotoconductive element of claim 1, wherein said titanylphthalocyanine is capable of forming different crystalline forms having different X-ray diffraction patterns.

4. The electrophotoconductive element of claim 1 comprising a mixture of titanylphthalocyanines having different crystalline forms having different X-ray diffraction patterns.

5. The electrophotoconductive element of claim 1 wherein said titanylphthalocyanine comprises mixture of Type I TiOPC and Type IV TiOPC.

6. The electrophotoconductive element of claim 5 wherein said Type I TiOPC indicates an X-ray diffraction having a strongest intensity of diffracted X-rays at about 26.0-27.0 degrees.

7. The electrophotoconductive element of claim 5 wherein said Type IV TiOPC indicates an X-ray diffraction having a strongest intensity of diffracted X-rays between 27.0-28.0 degrees.

8. The electrophotoconductive element of claim 1 positioned within an image forming apparatus.

9. The electrophotoconductive element of claim 1 positioned within a printer cartridge.

10. An electrophotographic photoconductor system comprising an electroconductive support, a charge generation layer, a charge transport layer and a source of laser light having a wavelength of about 350-500 nm, said charge transport layer exhibiting light transmitting properties with respect to said laser light wherein said charge generation layer comprises a titanylphthalocyanine (TiOPC) photoconductor capable of absorbing said laser light.

11. The system of claim 10 wherein said charge transport layer transmits 25% or more of said laser light.

12. The system of claim 10 wherein said titanylphthalocyanine comprises a mixture of Type I TiOPC and Type IV TiOPC.

13. The system of claim 10 wherein said Type I TiOPC indicates an X-ray diffraction having a strongest intensity of diffracted X-rays at about 26.0-27.0 degrees.

14. The system of claim 10 wherein said Type IV TiOPC indicates an X-ray diffraction having a strongest intensity of diffracted X-rays between 27.0-28.0 degrees.

15. The system of claim 10 positioned within an image forming apparatus.

16. The system of claim 10 positioned within a printer cartridge.

17. A method of forming an image in an electrophotographic device comprising the steps of:
(a) providing an electrophotoconductive element comprising an electroconductive support, a charge generation layer and a charge and a charge transport layer wherein said charge generation layer comprises a titanylphthalocyanine (TiOPC) photoconductor capable of absorbing laser light having a wavelength of about 350-500 nm; and
(b) charging the element and exposing to laser light restricted to a wavelength of about 350-500 nm and forming an electrostatic latent image.

18. The method of claim 17 wherein said TiOPC comprises Type IV TiOPC.

19. The method of claim 17 wherein said charge transport layer transmits 25% or more of said laser light.

20. The method of claim 17 where said method is carried out in an image forming apparatus.
